(19) United States
(12) Patent Application Publication
(10) Pub. No.: US 2025/0006311 A1
(43) Pub. Date: Jan. 2, 2025

(54) METHOD AND SYSTEM TO COMPUTE ABUNDANCE HISTOGRAM IN A PRIVACY PRESERVING MANNER

(71) Applicant: Tata Consultancy Services Limited, Mumbai (IN)

(72) Inventors: HARIKA NARUMANCHI, Hyderabad (IN); NITESH EMMADI, Hyderabad (IN); DIVYESH SAGLANI, Pune (IN); RAJAN MINDIGAL ALASINGARA BHATTACHAR, Bangalore (IN); NAVEEN SIVADASAN, Hyderabad (IN)

(73) Assignee: Tata Consultancy Services Limited, Mumbai (IN)

(21) Appl. No.: 18/750,734

(22) Filed: Jun. 21, 2024

(30) Foreign Application Priority Data
Jun. 27, 2023 (IN) 202321043119

(51) Int. Cl. G16B 50/40

(52) U.S. Cl. CPC G16B 50/40

(57) ABSTRACT

With the growing use of large-scale datasets containing participant's genomics and clinical data for research and studies purposes, it is important to ensure the privacy of the participant by generating a secure representation of genome data observation. Embodiments herein provide a system and method to perform DNA analysis on an encrypted genome without the need for decryption and ensure user's genome privacy. Herein, a stream of input genome is divided into chunks called as the sketched, where each k-mer in the sketch is hashed to get a sampling level and a counter value for the k-mer in a sketch. A privacy enhancing technique is designed over the representations using a Fully Homomorphic Encryption (FHE). Genomic data is analyzed with identified efficient algorithms for real world deployment in encrypted domain.

### PRIORITY CLAIM

This U.S. patent application claims priority under 35 U.S.C. § 119 to Indian application No. 202321043119, filed on Jun. 27, 2023. The entire contents of the aforementioned application are incorporated herein by reference.

### TECHNICAL FIELD

The disclosure herein generally relates to the field of preserving privacy of a genome, and, more particularly, to a method and system for computing k-mer abundance histogram on fully homomorphic encrypted genomic data in a privacy preserving manner.

### BACKGROUND

A genome is an organism's complete set of Deoxyribonucleic Acid (DNA), that includes enormous genetic information. The genome is a collection of DNA molecules storing genetic information in a cell. It can be represented as a set of long strings over the alphabet (each string corresponding to one chromosome). In the DNA sequencing experiment, many reads are produced from the genome. These reads are short substrings obtained from random locations of the genome. Genome size and some other characteristics estimates are computed from a summary statistic of reads i.e., a k-mer abundance histogram. A k-mer is a substring of length exactly k and the histogram summarizes the number of occurrences of individual k-mers in the input set of reads. The histogram gives insights on the approximate total number of distinct k-mers and total number of k-mers with some multiplicity (frequency) while preserving the privacy of critical genomic data.

K-mer abundance histogram estimation is a critical constituent and has several applications in genome analysis. For instance, k-mer abundance information in sequence data is useful in read error correction, parameter estimation for genome assembly, digital normalization, sampling error rate etc. However, existing k-mer abundance estimation techniques do not address the problem of data privacy. Data privacy is important especially in fields like genome analysis which involve sensitive data of a subject such as genetic testing information like DNA or any personal health information, which when leaked results in irreversible damage for the subject. Further, with the growing use of large-scale datasets containing the subject's genomics and clinical data for research and studies purposes, it is important to ensure the privacy of the subject by generating a secure representation of genome data observation.

### SUMMARY

Embodiments of the present disclosure present technological improvements as solutions to one or more of the above-mentioned technical problems recognized by the inventors in conventional systems. For example, in one embodiment, a method for computing abundance histogram on a fully homomorphic encrypted genomic data in a privacy preserving manner is provided. The processor-implemented method comprises receiving, via an input/output interface, a genome sequence as an input, wherein the genome is a string of a plurality of nucleotides. The input genome is divided into a series of chunks defined as sketches. Further, the processor-implemented method comprises estimating one or more distinct nucleotide strings for the received input genome sequence and one or more nucleotide strings with multiplicity using a privacy preserving kmerlight technique. Each of the one or more distinct nucleotide strings are hashed in each of the one or more sketches using a randomized hash function. A multilevel sampling is performed for each nucleotide string which involves dividing these strings into subsets based on length of a prefix. Furthermore, the processor-implemented method comprises determining a sampling level and a counter value of each nucleotide string based on the hash value of the nucleotide. The sampling level and the counter value of each nucleotide string is encrypted. The encrypted sampling level and the counter value are sent to a predefined server. Wherein, the predefined server checks condition for collision for the encrypted input data to update the matrix using the encrypted sampling level and counter value.

Further, the processor-implemented method comprises determining an optimal sampling level and associated optimal counter value for each multiplicity in the matrix. Wherein the optimal sampling level is the level which has the most occurrence of a multiplicity in the matrix. Further, an approximation of the one or more distinct nucleotide strings are determined for the given input genome and the one or more distinct nucleotide strings with a certain multiplicity based on the determined optimal sampling level and an associated optimal counter level. Finally, computing an abundance histogram on a fully homomorphic encrypted genomic data in privacy preserving manner based on the determined approximation of the total number of distinct nucleotide strings.

In another aspect, a system for computing abundance histogram on fully homomorphic encrypted genomic data in a privacy preserving manner is provided. The system comprises a memory storing a plurality of instructions and one or more Input/Output (I/O) interfaces to receive a genome sequence as an input, wherein the genome is a string of a plurality of nucleotides. Further, the system comprises one or more hardware processors coupled to the memory via the one or more I/O interfaces, wherein the one or more hardware processors are configured by the instructions to divide the string of the plurality of nucleotides into one or more sketches based on a predefined sketch size. Further, the one or more hardware processors are configured to estimating, via the one or more hardware processors, one or more distinct nucleotide strings for the received input genome sequence and one or more nucleotide strings with multiplicity using a privacy preserving kmerlight technique. Furthermore, the one or more hardware processors are configured to hash each of the one or more distinct nucleotide strings in each of the one or more sketches using a randomized hash function, wherein the hash value is used to determine a sampling level and a counter value of nucleotide string.

Further, the one or more hardware processors are configured to perform a multilevel sampling for each nucleotide string which involves dividing these strings into subsets based on length of a prefix, wherein the prefix comprises one or more trailing zeros and one or more leading zeros in the hash value of nucleotide. Further, the one or more hardware processors are configured to determining, via the one or more hardware processors, a sampling level and a counter value of each nucleotide string based on the hash value of the nucleotide, wherein a matrix of the sampling level and counter value is prepared for each of the one or more sketches. The matrix (one matrix created for the whole genome) is updated using the sampling level and counter value for each nucleotide string in each sketch. Furthermore, the one or more hardware processors are configured to encrypting, via the one or more hardware processors, the sampling level, and the counter value of each nucleotide string, wherein the encrypted sampling level and the counter value is sent to a predefined server, wherein the predefined server checks condition for collision for the encrypted input data to update the matrix of the encrypted sampling level and counter value, where in the matrix also called as sketch matrix is also encrypted.

Furthermore, the one or more hardware processors are configured to determine an encrypted optimal sampling level and associated encrypted optimal counter value for each multiplicity from the encrypted matrix, wherein the optimal sampling level is the level which has the most occurrence of a multiplicity in the matrix. Furthermore, the one or more hardware processors are configured to determine an encrypted approximation of the one or more distinct nucleotide strings for the given input genome and the one or more distinct nucleotide strings with a certain multiplicity based on the determined encrypted optimal sampling level and the associated encrypted optimal counter level. Finally, the one or more hardware processors are configured to compute an abundance histogram on a fully homomorphic encrypted genomic data in privacy preserving manner based on the determined approximation of the total number of distinct nucleotide strings.

In yet another aspect, there are provided one or more non-transitory machine-readable information storage mediums comprising one or more instructions, which when executed by one or more hardware processors causes a method for computing abundance histogram on a fully homomorphic encrypted genomic data in a privacy preserving manner is provided. The processor-implemented method includes receiving, via an input/output interface, a genome sequence as an input, wherein the genome is a string of a plurality of nucleotides. Furthermore, the processor-implemented method comprises estimating one or more distinct nucleotide strings for the received input genome sequence and one or more nucleotide strings with multiplicity using a privacy preserving kmerlight technique. Each of the one or more distinct nucleotide strings are hashed in each of the one or more sketches using a randomized hash function. A multilevel sampling is performed for each nucleotide string which involves dividing these strings into subsets based on length of a prefix. Further, the processor-implemented method comprises determining a sampling level and a counter value of each nucleotide string based on the hash value of the nucleotide. The sampling level and the counter value of each nucleotide string is encrypted. The encrypted sampling level and the counter value are sent to a predefined server. Wherein, the predefined server checks condition for collision for the encrypted input data to update the matrix of the sampling level and counter value.

Further, the processor-implemented method comprises determining an optimal sampling level and associated optimal counter value for each multiplicity in the matrix. Wherein the optimal sampling level is the level which has the most occurrence of a multiplicity in the matrix. Further, an approximation of the one or more distinct nucleotide strings are determined for the given input genome and the one or more distinct nucleotide strings with a certain multiplicity based on the determined optimal sampling level and an associated optimal counter level. Finally, computing an abundance histogram on a fully homomorphic encrypted genomic data in privacy preserving manner based on the determined approximation of the total number of distinct nucleotide strings.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, explain the disclosed principles:

FIG. 1 illustrates an exemplary system for computing abundance histogram on fully homomorphic encrypted genomic data in a privacy preserving manner, according to some embodiments of the present disclosure.

FIG. 2 is a flow diagram to illustrate an overview of privacy preserving genomics, according to some embodiments of the present disclosure.

FIGS. 3A and 3B (collectively referred as FIG. 3) are exemplary flow diagrams illustrating a processor-implemented method for computing abundance histogram on a fully homomorphic encrypted genomic data in a privacy preserving manner, according to some embodiments of the present disclosure.

FIG. 4 is a flow diagram to illustrate an overview of the kmerlight algorithm, according to some embodiments of the present disclosure.

FIGS. 5A and 5B (collectively referred to as FIG. 5) are schematic diagrams to illustrate a comparison of original Fᵢ function and the approximated Fᵢ function, according to some embodiments of the present disclosure.

FIGS. 6A and 6B (collectively referred as FIG. 6) are schematic diagrams to illustrate an error analysis in logarithmic approximation, according to some embodiments of the present disclosure.

### DETAILED DESCRIPTION

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the scope of the disclosed embodiments.

A genome is typically a string of nucleotides (Adenine (A), Cytosine (C), Guanine (G), and Thymine (T)). For example, ATGCGTCTC . . . G. Genomes are generally large biological sequence strings, and these are rendered into a stream of sub-strings of suitable length called k-mers and are used for computational genomics and sequence analysis. One such critical analysis is k-mer abundance estimation. Given an input genome sequence, k-mer abundance estimation algorithm provides estimations such as the total number of distinct k-mers in the sequence and total number of k-mers with frequency/multiplicity i (referred to as Fᵢ). There are several approaches in literature that can compute these estimates. However, these approaches do not address the privacy of the genome sequences.

Embodiments herein provide a method and system for computing abundance histogram on a fully homomorphic encrypted genomic data in a privacy preserving manner. The system is configured to estimate abundance of nucleotide strings on the encrypted nucleotide strings using a privacy preserving kmerlight algorithm. Kmerlight is a streaming algorithm, which in general, processes a sequence of k-mers in a single pass using only a limited amount of memory and time. The kmerlight maintains an approximate summary, or a sketch, of the previously viewed k-mers and with each new k-mer the sketch is updated. When all the k-mers are processed, the sketch can be analyzed to provide the estimate of the k-mer abundance histogram. Kmerlight combines the techniques of sampling and hashing to maintain a sketch of k-mers and from the contents of the sketch computes an estimate of the histogram.

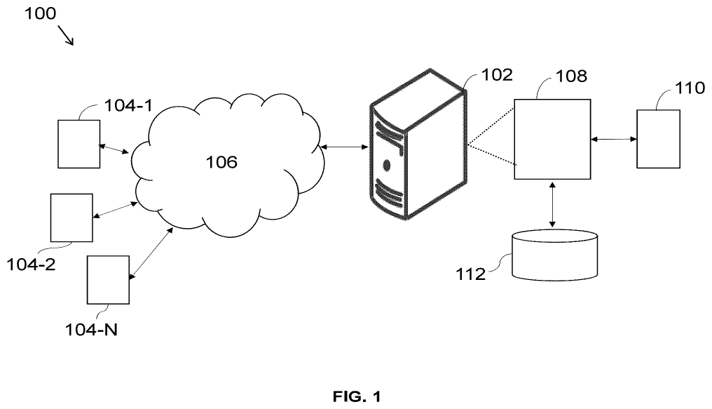

Referring now to the drawings, and more particularly to FIG. 1 through FIGS. 6, where similar reference characters denote corresponding features consistently throughout the figures, there are shown preferred embodiments, and these embodiments are described in the context of the following exemplary system and/or method.

FIG. 1 illustrates a block diagram of a system 100 for computing abundance histogram on a fully homomorphic encrypted genomic data in a privacy preserving manner, in accordance with an example embodiment. Although the present disclosure is explained considering that the system 100 is implemented on a server, it may be understood that the system 100 may comprise one or more computing devices 102, such as a laptop computer, a desktop computer, a notebook, a workstation, a cloud-based computing environment and the like. It will be understood that system 100 may be accessed through one or more input/output interfaces 104-1, 104-2 . . . 104-N, collectively referred to as I/O interface 104. Examples of the I/O interface 104 may include, but are not limited to, a user interface, a portable computer, a personal digital assistant, a handheld device, a smartphone, a tablet computer, a workstation, and the like. The I/O interface 104 is communicatively coupled to the system 100 through a network 106.

In an embodiment, the network 106 may be a wireless or a wired network, or a combination thereof. In an example, the network 106 can be implemented as a computer network, as one of the different types of networks, such as virtual private network (VPN), intranet, local area network (LAN), wide area network (WAN), the internet, and such. The network 106 may either be a dedicated network or a shared network, which represents an association of the different types of networks that use a variety of protocols, for example, Hypertext Transfer Protocol (HTTP), Transmission Control Protocol/Internet Protocol (TCP/IP), and Wireless Application Protocol (WAP), to communicate with each other. Further, network 106 may include a variety of network devices, including routers, bridges, servers, computing devices, storage devices. The network devices within network 106 may interact with the system 100 through communication links.

The system 100 supports various connectivity options such as BLUETOOTH®, USB, ZigBee, and other cellular services. The network environment enables connection of various components of the system 100 using any communication link including Internet, WAN, MAN, and so on. In an exemplary embodiment, the system 100 is implemented to operate as a stand-alone device. In another embodiment, the system 100 may be implemented to work as a loosely coupled device to a smart computing environment. Further, the system 100 comprises at least one memory with a plurality of instructions, one or more databases 112, and one or more hardware processors 108 which are communicatively coupled with the at least one memory to execute a plurality of modules 114 therein. The components and functionalities of the system 100 are described in further detail.

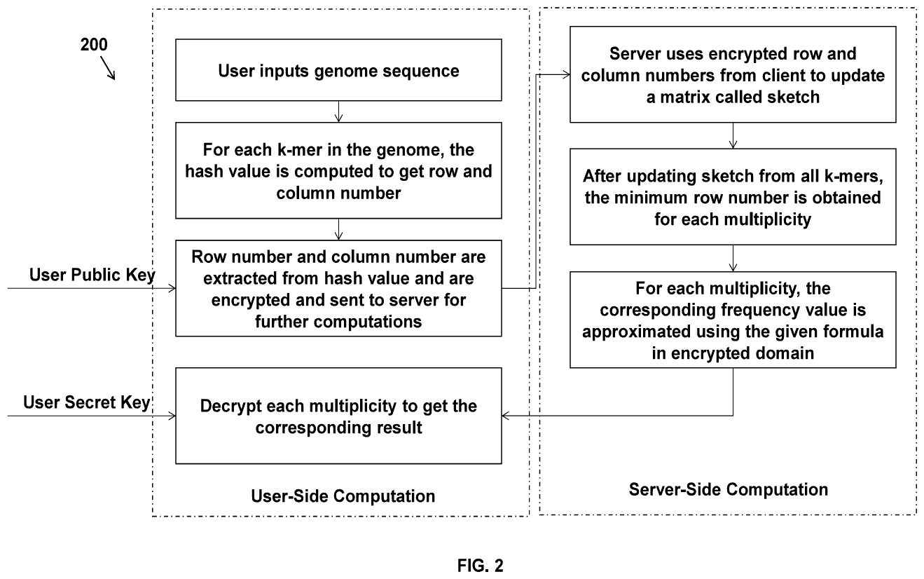

FIG. 2 is a schematic diagram 200 illustrating a privacy preserving genomics implemented by the system 100 of FIG. 1. A user wishes to use a DNA analysis service provided by a service provider; however the user does not want to disclose its genetic information to the service provider. Embodiments herein provide a system and method to perform DNA analysis on an encrypted genome without the need for decryption and ensure user's genome privacy. Herein, a stream of input genome is divided into chunks called as the sketches where each k-mer in the sketch is hashed to get a sampling level and a counter value for the k-mer in a sketch. The sampling level and counter value are used to update the k-mer position in a global matrix that is created for the single input genome. Using the global matrix, number of distinct k-mers and k-mers with multiplicity i are computed.

Further, a privacy enhancing technique is designed over the representations using a Fully Homomorphic Encryption (FHE). Genomic data is analyzed with identified efficient algorithms for real world deployment in encrypted domain. Analysis of k-mers, which are nucleotide strings of length k present in a genome sequence, is one of the fundamental operations in computational genomics. In particular, the problem of computing k-mer abundance estimation is considered in a genomic sequence.

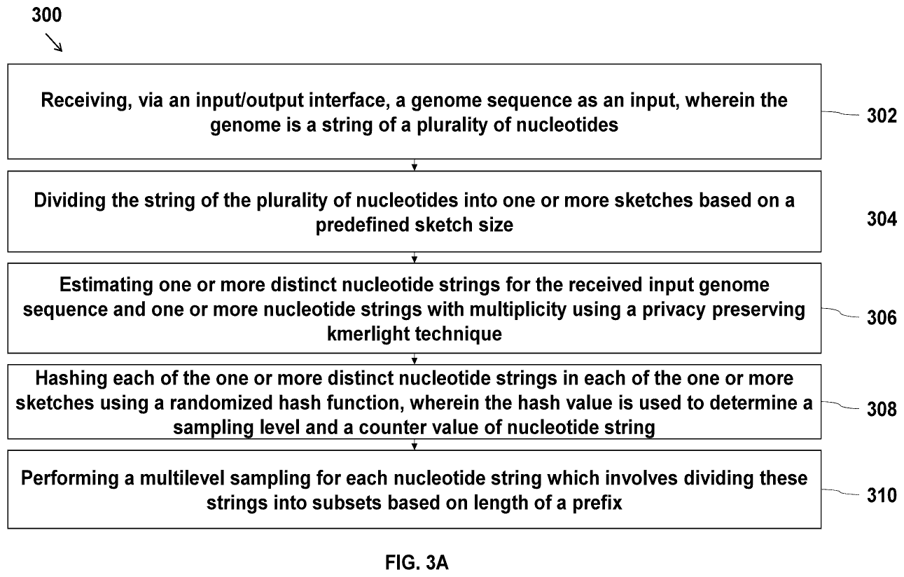

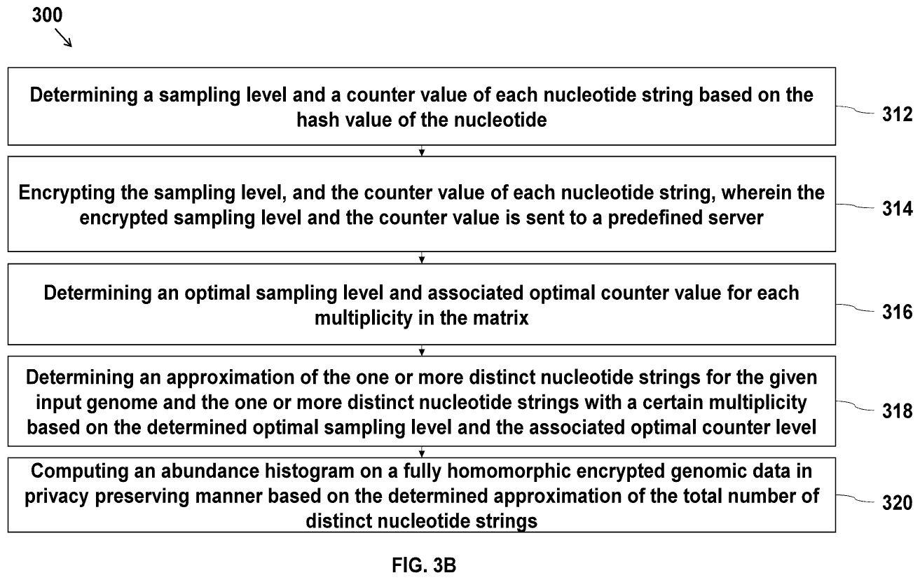

FIGS. 3A and 3B (collectively referred as FIG. 3) are flow diagrams illustrating a processor-implemented method 300 for computing abundance histogram on a fully homomorphic encrypted genomic data in a privacy preserving manner implemented by the system 100 of FIG. 1. Functions of the components of the system 100 are now explained with reference to FIG. 2 through steps of flow diagram in FIG. 3, according to some embodiments of the present disclosure.

Initially, at step 302 of the processor-implemented method 300, one or more hardware processors 108 are configured by the programmed instructions to receive a genome sequence as an input. The genome is a string of a plurality of nucleotides also called k-mers. Hereinafter, the string of a plurality of nucleotides is used interchangeably as k-mers.

At the next step 304 of the processor-implemented method 300, the one or more hardware processors 108 are configured by the programmed instructions to divide the string of the plurality of nucleotides into one or more sketches based on a predefined sketch size. In the sketching technique the input genome is subdivided into a series of chunks defined as sketches. Sketching is a method used in getting a simplified and summarized representation of large data sets that preserves important information while discarding the unnecessary details in a stream of data.

In one example, a user has a large stream of numbers, and the user wants to compute the average value of the entire list. The system is configured to generate reads from the bigger stream of numbers and create a sketch for each read that stores only a few representation numbers from the bigger list. The system can compute the average of each sketch and use these averages to compute the overall average that can provide a rough average estimate for the entire list.

Each sketch has a state matrix of a level and a counter with the size which is configurable such as level 64 and the counter=2²⁰. Each cell of the state matrix is updated. Further, the level number and counter value for each k-mer is obtained from the hash function in a sketch. The cell corresponding to this level number and counter number is then updated for the state matrix after checking if there exists no collision, where collision is a property where two k-mers have same level and counter number.

At the next step 306 of the processor-implemented method 300, the one or more hardware processors 108 are configured by the programmed instructions to estimate one or more distinct k-mers for the received input genome sequence and one or more k-mers with multiplicity using a privacy preserving kmerlight technique. The one or more distinct k-mers i.e., F₀ is computed as follows:

$${F}_{0} = {2}^{w - optimal} ⋆ \frac{log ( \frac{t - optimal}{R} )}{log ( 1 - \frac{1}{R} )} \qquad ( 1 )$$

wherein, w is the level number, which ranges from 0 . . . 64, w-optional is a level that has the number of counters with maximum count with a multiplicity i, t is the counter value which ranges from 0 . . . 2²⁰, and t-optimal is computed as max (number of levels—number of columns with multiplicity i). All the F₀ and Fᵢ are performed in encrypted domain, wherein t-optimal, w-optimal, F₀ and Fᵢ are encrypted and r is unencrypted and constant. The one or more k-mers with multiplicity i is computed as follows:

$${F}_{i} = \frac{{2}^{w - optimal} * ( ( \frac{t - 1}{R} ) * t - optimal )}{{( 1 - \frac{1}{R} )}^{{2}^{\frac{{F}_{0}}{w - optimal}}}} \qquad ( 2 )$$

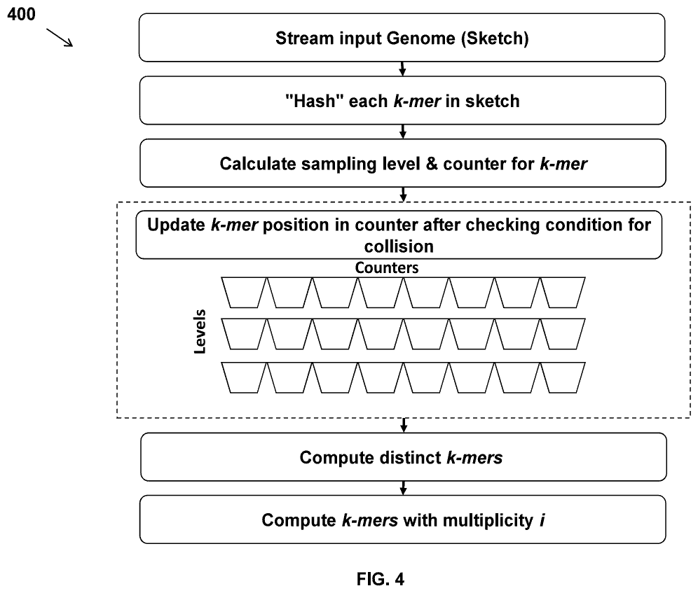

The one or more distinct k-mers are estimated using approximation formula provided, and to approximate nucleotides with multiplicity i, an optimal sampling level and counter number is determined from the sketch matrix and these numbers are used in the approximation formula to get the result as shown in FIG. 4. The optimal sampling level for a multiplicity i is the lowest level where maximum number of k-mers with multiplicity i are present. The optimal counter value is the maximum number of counters with multiplicity i from all levels.

At the next step 308 of the processor-implemented method 300, the one or more hardware processors 108 are configured by the programmed instructions to hash each of the one or more distinct k-mers in each of the one or more sketches using a randomized hash function. Wherein the hash value is used to determine a sampling level and a counter value of k-mer. Each input k-mer is hashed using a randomized hash function: hash_val←H(k-mer).

Each k-mer can be hashed using any secure randomized hash function. In one aspect, the hash function used by kmerlight algorithm is murmur3 hash. It is non-cryptographic hash function suitable for general hash-based lookup table. Murmur3 hash yields a 32-bit or 128-bit hash values. It is optimized based on the system architecture.

At the next step 310 of the processor-implemented method 300, the one or more hardware processors 108 are configured by the programmed instructions to perform a multilevel sampling for each nucleotide string which involves dividing these strings into subsets based on length of a prefix, wherein the prefix comprises one or more trailing zeros and one or more leading zeros in the hash value of nucleotide. In multi-level sampling, k-mers are segregated into levels, based on the length of the prefix say w, of hash values. For each level w, counters are maintained that determine the number of times each k-mer has occurred. For example,
sampling level (w)=1+number of trailing zeros in binary representation of hash;

$$counter value = floor ( \frac{x}{u} ) mod r ,$$

where r and u are predefined constants with values by the user. Wherein, r is sketching size and u is any constant value.
x=hash value/2^{w} where w is the sampling level.

At the next step 312 of the processor-implemented method 300, the one or more hardware processors 108 are configured by the programmed instructions to determine a sampling level and a counter value of each nucleotide string based on the hash value of the nucleotide, wherein a matrix of the sampling level and counter value is prepared for each of the one or more sketches. In the hash value for a k-mer obtained from murmur3 hash function, the number of trailing zeros in the hash value will give the sampling level number. The counter number is computed as:
x=hash value/2^{w}, where w is the sampling level obtained from above step and
counter number=floor (x/u) mod r, where r and u are predefined constants with values by the user.

At the next step 314 of the processor-implemented method 300, the one or more hardware processors 108 are configured by the programmed instructions to encrypt the sampling level and the counter value of each nucleotide string. The encrypted sampling level and the counter value is sent to a predefined server, wherein the predefined server checks condition for collision for the encrypted input data to update the matrix of the sampling level and counter value.

Note that the state-of-the-art data structures can handle only encryption of larger integers by increasing the parameter size. In data heavy applications, increasing the parameter size will make the application inefficient. In the present disclosure, a data structure called BigCipher is suitable for encrypting large integers, with a minimal parameter set. The BigCipher is like a big integer structure, a radix-based implementation in base b, which can accommodate large integer encryption. The BigCipher is designed to overcome the limitations existing in the current FHE library, that can only accommodate encryption of short integers. This data structure is especially important in the applications dealing with streaming inputs of larger sizes that need to be encrypted.

Further, the one or more hardware processors 108 are configured by the programmed instructions to approximate logarithm and exact computation of exponentiation of 2 in encrypted domain which can compute exponentiation of large integers without increasing the parameter set. This exponentiation function uses the idea of shifting. Usually, in FHE libraries, when exponentiation is computed for larger integers, the parameter sets need to be increased, which in turn impacts the performance adversely. This hinders performance and becomes inefficient as the size of integer grows. However, in the present disclosure, the function can compute exponentiation of large integers without increasing the parameter sets. The input is encoded using base-4 encoding and independently encrypted into a BigCipher data structure. Herein, an encrypted index of the first non-zero coefficient of the input in the BigCipher is extracted. For example, there is a number n=123, representation of n in base ‘4’ is shown below (size of 16 bits, which is enough to support 4¹⁶−1 numbers):
123=64+48+8+3=0000000000001323, each bit of the above representation is stored in the form of a vector, 0000010100101011 which as a whole represents a BigCipher.

For FHE, polynomial approximation is used and then approximation is applied. To compute non-linear function over FHE, the state-of-the-art techniques uses polynomial approximations like taylor-series approximation technique. The main drawback of this technique is that it is required to know the range of input, because the input has to be scaled to a smaller range for more accurate approximations. One such use case is logarithm approximation, where input is needed to be scaled down to (0,1]. Using bigCipher, the system doesn't need to scale down the input however large it may be and yet the system can still compute logarithm approximation with an average error of 0.05 and maximum error of 0.25. It is also important to note that this logarithm approximation algorithm provided by us is applied with a small FHE parameter set and provides a much better accuracy than polynomial approximation methods for large integers. It would be appreciated if it could work with a small FHE parameter set and is accurate for large integers. Logarithmic approximation with an average error of 0.05 and a maximum error of 0.25.

At the next step 316 of the processor-implemented method 300, one or more hardware processors 108 are configured by the programmed instructions to determine an optimal sampling level and associated optimal counter value for each multiplicity in the matrix. The optimal sampling level is the level which has the most occurrence of a multiplicity in the matrix.

To compute the optimal sampling level the following steps are performed. w-optimal is the optimal sampling level and t-optimal is the optimal counter value with initial value as 0. t is used to compute t-optimal, wherein t=number of k-mers with multiplicity i present in a row. For each sampling level w, if t>t-optimal, w-optimal=w, t-optimal=t. Using these last t-optimal and w-optimal values, the system is configured to compute the F₀ and Fᵢ values.

At the next step 318 of the processor-implemented method 300, the one or more hardware processors 108 are configured by the programmed instructions to determine an approximation of the one or more distinct k-mers for the given input genome and the one or more k-mers with a certain multiplicity based on the determined optimal sampling level and an associated optimal counter level as shown in Table 1.

TABLE 1

| | | Time for Plain |
|---|---|---|
| | Time for | Computation (using |
| Operation | BigCipher | direct operations) |

| | | | |
|---|---|---|---|
| Addition | 583.19 | ms | 230.0 ns |
| Logarithm | 15.41 | s | 60.0 ns |
| Exponentiation | 16.67 | s | 70.0 ns |
| Mux | 1.27 | s | 60.0 ns |
| Binary Multiplication | 112.97 | ms | 60.0 ns |
| Number Comparison | 3.36 | s | 70.0 ns |

Finally, at the last step 320 of the method 300, the one or more hardware processors 108 are configured by the programmed instructions to compute an abundance histogram on a fully homomorphic encrypted genomic data in privacy preserving manner based on the determined approximation of the total number of distinct nucleotide strings. Fully Homomorphic Encryption (FHE) enables computations on encrypted data without the need for decryption, thereby preserving privacy of the input data. For a set of FHE ciphertext corresponding to a set of plaintexts, any arbitrary function can be evaluated without revealing the plaintexts. FHE supports addition and multiplication as primitive operations:

$$Enc ( a + b ) = Enc ( a ) + Enc ( b ) \qquad ( 3 )$$

$$Enc ( a + b ) = Enc ( a ) + Enc ( b ) \qquad ( 4 )$$

A public key FHE scheme ξ consists of an additional eval ξ along with the usual (KeyGen _{ξ}, Enc _{ξ}, Dec _{ξ}) from any other public key scheme. Eval & is the evaluation algorithm used for computations on encrypted data. This algorithm takes as input a polynomial expression P and a set of ciphertext sc={C₀, C₁, . . . , Cₙ} as inputs to P. The input output of eval _{ξ} satisfies following equation:

$${Dec}_{ξ} ( {Eval}_{ξ} ( P , c , pk ) , sk ) = P ( {Dec}_{ξ} ( c , sk ) \qquad ( 5 )$$

To improve the efficiency of the homomorphic operations and to reduce space complexity, one can leverage homomorphic batching technique where multiple plaintexts is batched into a single ciphertext. On this batched ciphertext, the operations can be performed on component wise plaintexts and can be executed parallelly in single Instruction Multiple Data (SIMD) manner.

For a radix-based implementation of a number in base b, the logarithm computes the log value of the number in base ‘b’. Logarithm is expressed as sum of mantissa and exponent, where mantissa is the integer part and exponent are the decimal part. To compute mantissa, the index of the last non-zero coefficient of the radix representation of number is found in zero based indexing. To compute the decimal, the values from the last non-zero index are copied to evaluate it as in base b representation of decimal number. To compute log (n) in base 4:
log(x)=mantissa+exponent
mantissa=index of first non-zero index from left (or index of last non-zero element from right)=3
exponent=number from the first non-zero element represented as decimal representation for base b=1323000000000000
the base 10 representation for above result is=3+(1*(¼)+3*( 1/16)+2*( 1/64)+ . . . )=3.48
actual log value for log (123)/log (4)=3.471

### EXPERIMENT

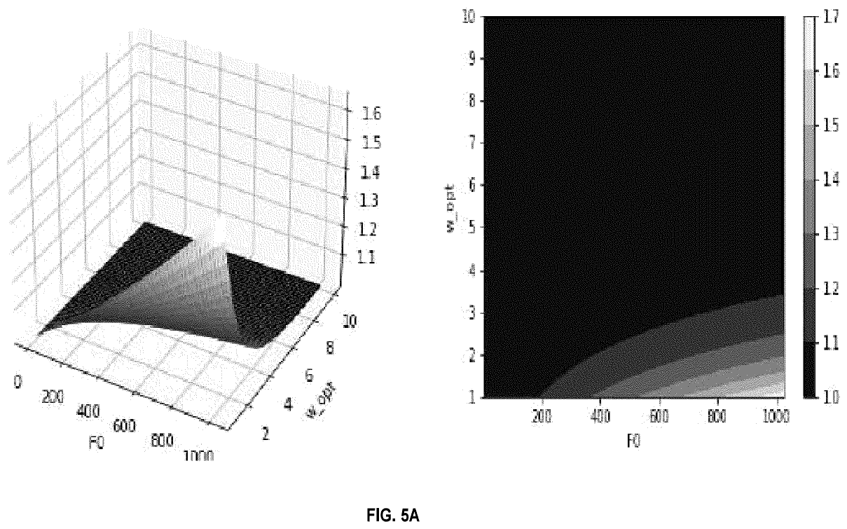

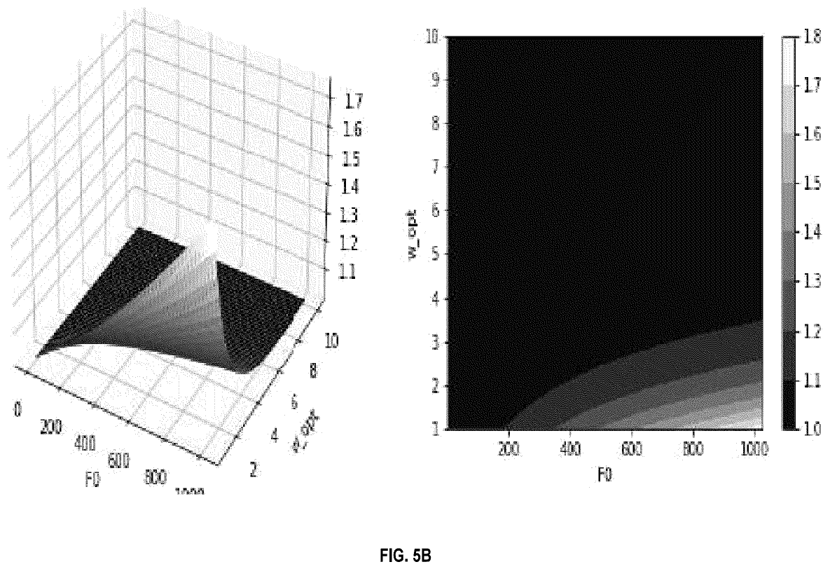

FIGS. 5A and 5B are schematic diagrams to illustrate a comparison of original Fᵢ function and the approximated Fᵢ function, according to some embodiments of the present disclosure. The graph compares the original Fᵢ equation in kmerlight algorithm with the approximation algorithm in the privacy preserving kmerlight. The approximation equation has a minimal loss and accuracy is above 96%.

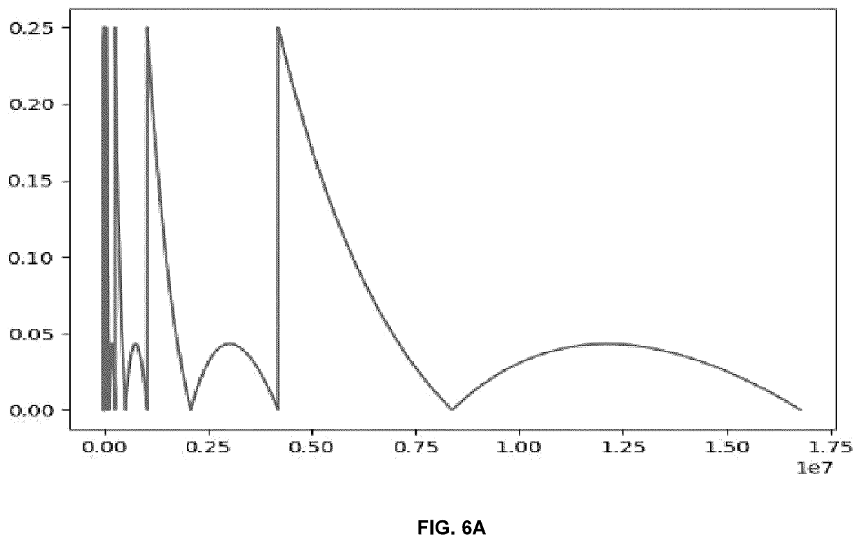

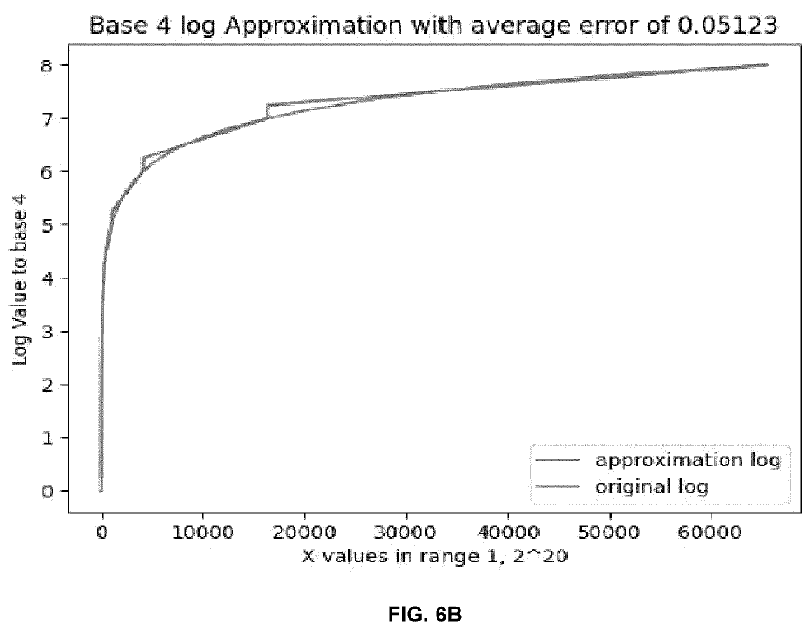

FIGS. 6A and 6B (collectively referred as FIG. 6) are schematic diagrams to illustrate an error analysis in logarithmic approximation, according to some embodiments of the present disclosure. In the FIG. 6A, the graph provides a range of possible error values for the logarithmic approximation function. The X-axis represents the input values in the range 1 to 2²⁰ and Y-axis represents the error between original logarithm and approximated logarithm. FIG. 6B provides the comparison of the original logarithm with the approximation of logarithm. The X-axis represents the input values in the range 1 to 2²⁰ and Y-axis represents the output of logarithmic computation with an average error of 0.05.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments of the present disclosure herein address unresolved problems of data privacy especially in genome analysis which involve sensitive data of a subject such as genetic testing information. This disclosure relates generally to a method and system for computing abundance histogram on fully homomorphic encrypted genomic data in a privacy preserving manner. privacy enhancing technique is designed over the representations using a Fully Homomorphic Encryption. Genomic data is analyzed with identified efficient algorithms for real world deployment in encrypted domain. Analysis of k-mers, which are nucleotide strings of length k present in a genome sequence, is one of the fundamental operations in computational genomics. In particular, the problem of computing k-mer abundance estimation is considered in a genomic sequence.

It is to be understood that the scope of the protection is extended to such a program and in addition to a computer-readable means having a message therein; such computer-readable storage means contain program-code means for implementation of one or more steps of the method, when the program runs on a server or mobile device or any suitable programmable device. The hardware device can be any kind of device which can be programmed including e.g., any kind of computer like a server or a personal computer, or the like, or any combination thereof. The device may also include means which could be e.g., hardware means like e.g., an application-specific integrated circuit (ASIC), a field-programmable gate array (FPGA), or a combination of hardware and software means, e.g., an ASIC and an FPGA, or at least one microprocessor and at least one memory with software processing components located therein. Thus, the means can include both hardware means, and software means. The method embodiments described herein could be implemented in hardware and software. The device may also include software means. Alternatively, the embodiments may be implemented on different hardware devices, e.g., using a plurality of CPUs.

The embodiments herein can comprise hardware and software elements. The embodiments that are implemented in software include but are not limited to, firmware, resident software, microcode, etc. The functions performed by various components described herein may be implemented in other components or combinations of other components. For the purposes of this description, a computer-usable or computer readable medium can be any apparatus that can comprise, store, communicate, propagate, or transport the program for use by or in connection with the instruction execution system, apparatus, or device.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope of disclosed embodiments being indicated by the following claims.

What is claimed is:

1. A processor-implemented method, comprising:
receiving, via an input/output interface, a genome sequence as an input, wherein the genome is a string of a plurality of nucleotides;
dividing, via one or more hardware processors, the string of the plurality of nucleotides into one or more sketches based on a predefined sketch size;
estimating, via the one or more hardware processors, one or more distinct nucleotide strings for the received input genome sequence and one or more nucleotide strings with multiplicity using a privacy preserving kmerlight technique;
hashing, via the one or more hardware processors, each of the one or more distinct nucleotide strings in each of the one or more sketches using a randomized hash function, wherein a hash value is used to determine a sampling level and a counter value of nucleotide string;
performing, via the one or more hardware processors, a multilevel sampling for each nucleotide string which involves dividing these strings into subsets based on length of a prefix;
determining, via the one or more hardware processors, a sampling level and a counter value of each nucleotide string based on the hash value of the nucleotide;
encrypting, via the one or more hardware processors, the sampling level, and the counter value of each nucleotide string, wherein the encrypted sampling level and the counter value is sent to a predefined server;
determining, via the one or more hardware processors, an optimal sampling level and an associated optimal counter value for each multiplicity in the matrix;
determining, via the one or more hardware processors, an approximate count of the one or more distinct nucleotide strings for the given input genome and the one or more distinct nucleotide strings with a certain multiplicity based on the determined optimal sampling level and the associated optimal counter level; and
computing, via the one or more hardware processors, an abundance histogram on a fully homomorphic encrypted genomic data in privacy preserving manner based on the determined approximation of the total number of distinct nucleotide strings.

2. The processor-implemented method of claim 1, wherein the prefix comprises one or more trailing zeros and one or more leading zeros in the hash value of nucleotide.

3. The processor-implemented method of claim 1, wherein a matrix of the sampling level and counter value is prepared for each of the one or more sketches.

4. The processor-implemented method of claim 1, wherein the matrix is updated using the sampling level and counter value for each nucleotide string in each sketch.

5. The processor-implemented method of claim 1, wherein the predefined server checks condition for collision for the encrypted input data to update the matrix of the sampling level and counter value.

6. The processor-implemented method of claim 1, wherein the optimal sampling level is the level which has the most occurrence of a multiplicity in the matrix.

7. A system comprising:
a plurality of input/output interfaces to receive a genome sequence as an input, wherein the genome is a string of a plurality of nucleotides;
a memory in communication with the one or more hardware processors, wherein the one or more hardware processors are configured to execute programmed instructions stored in the memory to:
divide the string of the plurality of nucleotides into one or more sketches based on a predefined sketch size;
estimate one or more distinct nucleotide strings for the received input genome sequence and one or more nucleotide strings with multiplicity using a privacy preserving kmerlight technique;
hash each of the one or more distinct nucleotide strings in each of the one or more sketches using a randomized hash function, wherein a hash value is used to determine a sampling level and a counter value of nucleotide string;
perform a multilevel sampling for each nucleotide string which involves dividing these strings into subsets based on length of a prefix, wherein the prefix comprises one or more trailing zeros and one or more leading zeros in the hash value of nucleotide;
determine a sampling level and a counter value of each nucleotide string based on the hash value of the nucleotide, wherein a matrix of the sampling level and counter value is prepared for each of the one or more sketches;
encrypt the sampling level and the counter value of each nucleotide string, wherein the encrypted sampling level and the counter value is sent to a predefined server, wherein the predefined server checks condition for collision for the encrypted input data to update the matrix of the sampling level and counter value;
determine an optimal sampling level and an associated optimal counter value for each multiplicity in the matrix, wherein the optimal sampling level is the level which has the most occurrence of a multiplicity in the matrix;
determine an approximation of the one or more distinct nucleotide strings for the given input genome and the one or more distinct nucleotide strings with a certain multiplicity based on the determined optimal sampling level and the associated optimal counter level; and
compute an abundance histogram on a fully homomorphic encrypted genomic data in privacy preserving manner based on the determined approximation of the total number of distinct nucleotide strings.

8. The system of claim 7, wherein the prefix comprises one or more trailing zeros and one or more leading zeros in the hash value of nucleotide.

9. The system of claim 7, wherein a matrix of the sampling level and counter value is prepared for each of the one or more sketches.

10. The system of claim 7, wherein the matrix is updated using the sampling level and counter value for each nucleotide string in each sketch.

11. The system of claim 7, wherein the predefined server checks condition for collision for the encrypted input data to update the matrix of the sampling level and counter value.

12. The system of claim 7, wherein the optimal sampling level is the level which has the most occurrence of a multiplicity in the matrix.

13. One or more non-transitory machine-readable information storage mediums comprising one or more instructions which when executed by one or more hardware processors cause:
receiving, via an input/output interface, a genome sequence as an input, wherein the genome is a string of a plurality of nucleotides;
dividing, via one or more hardware processors, the string of the plurality of nucleotides into one or more sketches based on a predefined sketch size;
estimating, via the one or more hardware processors, one or more distinct nucleotide strings for the received input genome sequence and one or more nucleotide strings with multiplicity using a privacy preserving kmerlight technique;
hashing, via the one or more hardware processors, each of the one or more distinct nucleotide strings in each of the one or more sketches using a randomized hash function, wherein a hash value is used to determine a sampling level and a counter value of nucleotide string;
performing, via the one or more hardware processors, a multilevel sampling for each nucleotide string which involves dividing these strings into subsets based on length of a prefix;
determining, via the one or more hardware processors, a sampling level and a counter value of each nucleotide string based on the hash value of the nucleotide;
encrypting, via the one or more hardware processors, the sampling level, and the counter value of each nucleotide string, wherein the encrypted sampling level and the counter value is sent to a predefined server;
determining, via the one or more hardware processors, an optimal sampling level and an associated optimal counter value for each multiplicity in the matrix;
determining, via the one or more hardware processors, an approximate count of the one or more distinct nucleotide strings for the given input genome and the one or more distinct nucleotide strings with a certain multiplicity based on the determined optimal sampling level and the associated optimal counter level; and
computing, via the one or more hardware processors, an abundance histogram on a fully homomorphic encrypted genomic data in privacy preserving manner based on the determined approximation of the total number of distinct nucleotide strings.

14. The one or more non-transitory machine-readable information storage mediums of claim 13, wherein the prefix comprises one or more trailing zeros and one or more leading zeros in the hash value of nucleotide.

15. The one or more non-transitory machine-readable information storage mediums of claim 13, wherein a matrix of the sampling level and counter value is prepared for each of the one or more sketches.

16. The one or more non-transitory machine-readable information storage mediums of claim 13, wherein the matrix is updated using the sampling level and counter value for each nucleotide string in each sketch.

17. The one or more non-transitory machine-readable information storage mediums of claim 13, wherein the predefined server checks condition for collision for the encrypted input data to update the matrix of the sampling level and counter value.

18. The one or more non-transitory machine-readable information storage mediums of claim 13, wherein the optimal sampling level is the level which has the most occurrence of a multiplicity in the matrix.
